SEQUENCE LISTING (1 sequence)

SEQ ID NO: 1 - DNA, 1411 bp, synthetic construct
  source 1..1411: mol_type = other DNA
ctcgatagagagcttatagactgcagtcgacgagagattgcttagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctgaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagtcataaggagacattg

(19) United States
(12) Patent Application Publication
(10) Pub. No.: US 2026/0139221 A1
(43) Pub. Date: May 21, 2026

(54) Akkermansia muciniphila MG784 for alleviating constipation,promoting defecation, improving intestinal barrier function and intestinal immunity, and its uses

(71) Applicant: Mom's Garden GmbH, Saarbrücken (DE)

(72) Inventors: Jing LI, Saarbrücken (DE); Naeyoung KANG, Saarbrücken (DE)

(21) Appl. No.: 19/385,194

(22) Filed: Nov. 11, 2025

(30) Foreign Application Priority Data
Sep. 17, 2025 (CN) 202511332821.X

(51) Int. Cl. C12N 1/205; A61K 31/7004; A61K 31/7016; A61K 31/702; A61K 31/7034; A61K 31/7048; A61K 31/717; A61K 31/733; A61K 35/00; A61K 35/741; A61K 35/742; A61K 35/744; A61K 35/745; A61K 35/747; A61K 45/06; A61P 1/10; C12R 1/01

(52) U.S. Cl. CPC C12N 1/205; A61K 31/7004; A61K 31/7016; A61K 31/702; A61K 31/7034; A61K 31/7048; A61K 31/717; A61K 31/733; A61K 35/741; A61K 35/742; A61K 35/744; A61K 35/745; A61K 35/747; A61K 45/06; A61P 1/10; A61K 2035/115; C12R 2001/01

(57) ABSTRACT

The present invention relates to the field of probiotics, in particular to an Akkermansia muciniphila MG784 for alleviating constipation, promoting defecation, improving intestinal barrier function and intestinal immunity, and its uses. The Akkermansia muciniphila MG784 provided by the present invention has the deposition number DSM 35549. The Akkermansia muciniphila MG784 provided by the present invention can increase the levels of motilin (MTL), gastrin (GAS), and acetylcholine in the gastrointestinal tract, promote intestinal peristalsis, improve the small intestine propulsion rate, and ameliorate defecation time and fecal output. Furthermore, Akkermansia muciniphila MG784 can also alleviate colonic mucosal inflammation, improve the thickness of the digestive tract muscular layer, modulate the structure of the intestinal microbiota, significantly increase the abundance of beneficial bacteria, and decrease the abundance of harmful bacteria.

### CROSS REFERENCE TO RELATED APPLICATIONS

The present disclosure claims priority to Chinese Patent Application No. 202511332821.X, entitled “Akkermansia Muciniphila Mg784 For Alleviating Constipation, Promoting Defecation, Improving Intestinal Barrier Function And Intestinal Immunity, And Its Uses”, filed on Sep. 17, 2025, the entire content of which are incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 3,310 Byte XML file named “Sequence listing.xml,” dated Nov. 6, 2025.

### TECHNICAL FIELD

The present invention relates to the field of probiotics, in particular to an Akkermansia muciniphila for alleviating constipation, promoting defecation, improving intestinal barrier function and intestinal immunity, and its uses.

### BACKGROUND

Constipation, as a highly prevalent digestive system disorder, is characterized by difficulty in defecation, dry and hard stools, and reduced defecation frequency. This condition not only reduces patients' quality of life but also easily induces perianal diseases (such as hemorrhoids, anal fissures) and intestinal dysfunction. Current clinical interventions mainly include laxative drugs, dietary fiber supplementation, and biofeedback therapy, but they generally suffer from limitations such as variable efficacy, risk of drug dependence, and long-term use leading to electrolyte imbalance.

With breakthroughs in gut microbiome research, probiotic therapy has attracted much attention due to its ability to reshape the intestinal microbiota ecology and activate enteric neural motility. Probiotics refer to live microorganisms that, when administered in adequate amounts, confer a health benefit on the host. Substantial clinical evidence indicates that specific probiotic strains can effectively improve constipation through mechanisms such as secreting short-chain fatty acids and regulating enteric neurotransmitters, with safety superior to traditional drugs. However, conventional probiotic products are often limited by factors such as single strain selection, low survival rate in gastric acid environments, and weak intestinal colonization ability, resulting in clinical effects often falling short of expectations.

Against this backdrop, Akkermansia muciniphila, as a representative of next-generation probiotics, has gained prominence. This bacterium belongs to the phylum Verrucomicrobia and colonizes the intestinal mucus layer. It was first isolated from the human gut in 2004 and is named for its unique ability to degrade mucin. Rather than compromising the intestinal barrier, this distinctive mucin-based metabolism stimulates host cells to produce more and thicker mucus, thereby strengthening the intestinal physical barrier. Furthermore, current research has revealed that this bacterium exerts multiple physiological effects through the following mechanisms: Intestinal barrier repair: Metabolizing mucin produces compounds such as butyrate, which nourish colonic epithelial cells; Immune regulation: Increasing the thickness of the intestinal mucus layer and inhibiting the release of pro-inflammatory factors; Metabolic improvement: Enhancing insulin sensitivity and reducing fat accumulation; Lipid excretion: Decreasing fat absorption in both animals and humans, promoting lipid excretion, and reducing visceral fat accumulation; Constipation relief: Promoting intestinal peristalsis, accelerating content transit, and increasing fecal excretion. Its potential applications in obesity, diabetes, and inflammatory bowel disease have been validated by numerous trials, offering a new direction for the treatment of functional gastrointestinal disorders.

### SUMMARY

In view of the above-mentioned drawbacks of the prior art, the objective of the present invention is to provide an Akkermansia muciniphila for alleviating constipation and promoting defecation, and its uses, to solve the problems in the prior art.

To achieve the above objective and other related objectives, the present invention provides an Akkermansia muciniphila MG784, deposited under the German accession number DSM 35549, with a deposit date of Aug. 18, 2025, at the Leibniz Institute DSMZ-German Collection of Microorganisms and Cell Cultures Inhoffenstraße 7 B, 38124 Braunschweig, GERMANY, and also deposited under the Chinese accession number CGMCC No. 46459, with a deposit date of Jul. 3, 2025, at the China General Microbiological Culture Collection Center (CGMCC), Institute of Microbiology, Chinese Academy of Sciences, Beijing, China.

Preferably, the Akkermansia muciniphila MG784 comprises a DNA fragment having a similarity of 50% or more to the nucleotide sequence shown as SEQ ID No. 1.

The present invention also provides a composition comprising the aforementioned Akkermansia muciniphila MG784.

Preferably, the composition further comprises one or more selected from the group consisting of probiotic, prebiotic, vitamin, mineral, amino acid, polypeptide, emulsifier, thickener, sweetener, flavoring agent, stabilizers, and preservative.

The present invention also provides an acetylcholinesterase inhibitor comprising the aforementioned Akkermansia muciniphila MG784 or its fermentation product.

The present invention also provides the use of the aforementioned Akkermansia muciniphila MG784 or the fermentation product thereof, or the aforementioned composition or the fermentation product thereof, in the manufacture of a product for improving constipation, a product for enhancing immune function, a product for protecting the intestine, a product for regulating the intestinal microbiota, a product for oil-discharging or fat-reducing, an antioxidant product, an anti-inflammatory product, or an acetylcholinesterase inhibitor.

The present invention also provides a method for improving constipation, enhancing immune function, protecting the intestine, regulating the intestinal microbiota, discharging oil, reducing fat, providing antioxidant effects, providing anti-inflammatory effects, or inhibiting acetylcholinesterase activity, characterized in that the method comprises administering to a subject an effective amount of the aforementioned Akkermansia muciniphila MG784 or the aforementioned composition.

As described above, the Akkermansia muciniphila MG784 of the present invention for alleviating constipation, promoting defecation, improving intestinal barrier function and intestinal immunity, and its uses, have the following beneficial effects:

The Akkermansia muciniphila MG784 provided by the present invention can increase the levels of motilin (MTL), gastrin (GAS), and acetylcholine in the gastrointestinal tract, promote intestinal peristalsis, improve the small intestine propulsion rate, and ameliorate defecation time and fecal output. Furthermore, the Akkermansia muciniphila MG784 can also alleviate colonic mucosal inflammation, improve the thickness of the digestive tract muscular layer, modulate the structure of the intestinal microbiota, significantly increase the abundance of beneficial bacteria, and decrease the abundance of harmful bacteria.

### BRIEF DESCRIPTION OF THE DRAWINGS

FIG. 1 shows a schematic diagram of the acetylcholinesterase inhibition rate of the inactivated Akkermansia muciniphila MG784 of the present invention (Compared with the blank group and control group, p *<0.05).

FIG. 2 shows a schematic diagram of the effect of the Akkermansia muciniphila MG784 of the present invention on the small intestine propulsion rate in constipated model mice (Compared with the model group, p *<0.05, p **<0.01, p ***<0.001, p ****<0.0001).

FIGS. 3A-3C show a schematic diagram of the effect of the Akkermansia muciniphila strain MG784 of the present invention on the first black stool time, total number of fecal pellets in 6 h, and total weight of fecal pellets in 6 h in constipated model mice (Compared with the model group, p*<0.05, p **<0.01, p ***<0.001, p ****<0.0001).

FIGS. 4A-4C show a schematic diagram of the effect of the Akkermansia muciniphila MG784 of the present invention on the levels of motilin (MTL), gastrin (GAS), and colon acetylcholine content in constipated model mice (Compared with the model group, p*<0.05, p **<0.01, p ***<0.001, p ****<0.0001).

FIGS. 5A-5B show a schematic diagram of the effect of the Akkermansia muciniphila MG784 of the present invention on the histopathological staining results of colon tissue in constipated model mice (Compared with the model group, p *<0.05, p **<0.01, p ***<0.001, p ****<0.0001).

FIGS. 6A-6B shows a schematic diagram of the effect of the Akkermansia muciniphila strain MG784 of the present invention on the changes in the total amount of beneficial and harmful bacteria at the genus level in the intestinal microbiota of constipated model mice (Compared with the model group, p *<0.05, p **<0.01, p ***<0.001, p ****<0.0001).

### DETAILED DESCRIPTION OF THE EMBODIMENTS

The present invention provides an Akkermansia muciniphila MG784, deposited under the German accession number deposited under the accession number DSM 35549, with a deposit date of Aug. 18, 2025, at the Leibniz Institute DSMZ-German Collection of Microorganisms and Cell Cultures Inhoffenstraße 7 B, 38124 Braunschweig, GERMANY, and also deposited under the Chinese accession number CGMCC No. 46459, with a deposit date of Jul. 3, 2025, at the China General Microbiological Culture Collection Center (CGMCC), Institute of Microbiology, Chinese Academy of Sciences, Beijing, China.

In some specific embodiments, the Akkermansia muciniphila MG784 comprises a DNA fragment having 50% or more similarity to the nucleotide sequence set forth as SEQ ID NO: 1. Specifically, the Akkermansia muciniphila MG784 comprises a DNA fragment having a nucleotide sequence similarity of 50%, 55%, 60%, 65%, 70%, 75%, 80%, 82%, 84%, 86%, 88%, 90%, 91%, 92%, 93%, 94%, 95%, 96%, 97%, 98%, or 99% or more to SEQ ID NO: 1. Preferably, the Akkermansia muciniphila MG784 comprises a DNA fragment having a nucleotide sequence similarity of 90% or more to SEQ ID NO: 1; more preferably, it comprises a DNA fragment having a nucleotide sequence similarity of 99% or more to SEQ ID NO: 1. Specifically, the DNA fragment contained in the Akkermansia muciniphila MG784 may be obtained through nucleotide substitution, deletion, insertion, or homologous recombination of a nucleic acid fragment based on the DNA strand with the nucleotide sequence set forth as SEQ ID NO: 1.

The present invention also provides a composition comprising the aforementioned Akkermansia muciniphila MG784.

In some specific embodiments, the composition further comprises one or more selected from the group consisting of probiotic, prebiotic, vitamin, mineral, amino acid, polypeptide, emulsifier, thickener, sweetener, flavoring agent, stabilizer, and preservative.

Further, the probiotic can be selected from one or more of Bifidobacterium, Lactobacillus, Enterococcus, Bacillus, Clostridium, Saccharomyces, Weizmannia (formerly Bacillus), or Lactococcus. Preferably, the probiotic is selected from one or more of Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium longum, Bifidobacterium infantis, Bifidobacterium animalis, Bifidobacterium bifidum, Bifidobacterium adolescentis, Bifidobacterium lactis, Enterococcus faecalis, Enterococcus faecium, Lactococcus lactis, Streptococcus salivarius, Saccharomyces cerevisiae and Saccharomyces boulardii, Clostridium butyricum, Bacillus subtilis, Bacillus indicus, Bacillus licheniformis, Bacillus clausii, or Bacillus coagulans.

Further, the prebiotic can be one or more selected from the group consisting of inulin, inulin-type fructans, fructo-oligosaccharides, xylose, arabinose, arabinoxylans, ribose, galactose, rhamnose, cellobiose, fructose, lactose, salicin, sucrose, glucose, esculin, Tween 80, trehalose, maltose, mannose, melibiose, mucus or mucin, raffinose, fructo-oligosaccharides, galacto-oligosaccharides, or amino acids.

In some specific embodiments, the Akkermansia muciniphila strain MG784 has a concentration in the composition of at least 1×10⁹ CFU/g, at least 2×10⁹ CFU/g, at least 3×10⁹ CFU/g, at least 4×10⁹ CFU/g, at least 5×10⁹ CFU/g, at least 6×10⁹ CFU/g, at least 7×10⁹ CFU/g, at least 8×10⁹ CFU/g, at least 9×10⁹ CFU/g, at least 1×10¹⁰ CFU/g, at least 2×10¹⁰ CFU/g, at least 3×10¹⁰ CFU/g, at least 4×10¹⁰ CFU/g, at least 5×10¹⁰ CFU/g, at least 6×10¹⁰ CFU/g, at least 7×10¹⁰ CFU/g, at least 8×10¹⁰ CFU/g, at least 9×10¹⁰ CFU/g, or at least 1×10¹¹ CFU/g or more.

In some specific embodiments, the composition is selected from one or more of capsule, tablet, powder, granule, powder, oral liquid, drop, gummy, pressed candy, jelly, solid beverage, or fermented dairy product.

The present invention also provides an acetylcholinesterase inhibitor comprising the aforementioned Akkermansia muciniphila MG784 or its fermentation product.

The present invention also provides the use of the aforementioned Akkermansia muciniphila MG784 or the fermentation product thereof, or the aforementioned composition, or the fermentation product thereof, for improving constipation, a product for enhancing immune function, a product for protecting the intestine, a product for regulating the intestinal microbiota, a product for oil-discharging or fat-reducing, an antioxidant product, an anti-inflammatory product, or an acetylcholinesterase inhibitor.

In some specific embodiments, the product for improving constipation has one or more of the following functions:
Enhancing intestinal peristalsis;
Reducing defecation time;
Increasing fecal weight;
Increasing intestinal acetylcholine content.

In some specific embodiments, the product for improving constipation, the product for enhancing immune function, the product for protecting the intestine, the product for regulating the intestinal microbiota, the product for oil-discharging or fat-reducing, the antioxidant product, or the anti-inflammatory product is a food, health food, or pharmaceutical.

In some specific embodiments, the anti-inflammatory product is a product for anti-vaginitis; and/or, the product for enhancing immune function is a product for improving intestinal immunity; and/or, the product for protecting the intestine is a product for improving intestinal barrier function.

In some specific embodiments, the excipient acceptable for food, health food, or pharmaceuticals includes various excipients and diluents, which are not essential active ingredients and do not exhibit undue toxicity after administration. The excipient includes sterile water or physiological saline, stabilizers, excipients, antioxidants (ascorbic acid, etc.), buffers (phosphoric acid, citric acid, other organic acids, etc.), preservatives, surfactants (PEG, Tween, etc.), chelating agents (EDTA, etc.), or binders. The excipient also includes other low molecular weight polypeptides, serum albumin, glycine, glutamine, asparagine, arginine, polysaccharides, monosaccharides, mannitol, or sorbitol. The excipient for injectable aqueous solutions is selected from physiological saline, isotonic glucose solution, isotonic D-sorbitol solution, isotonic D-mannose solution, isotonic D-mannitol solution. The injectable aqueous solution includes a solubilizer. The solubilizer is selected from alcohols (ethanol), polyols (propylene glycol or PEG), and/or non-ionic surfactants (Tween 80 or HCO-50).

In some specific embodiments, the use comprises administering to a subject an effective amount of aforementioned Akkermansia muciniphila MG784, or its fermentation product or the aforementioned composition, or its fermentation product.

In some specific embodiments, the subject is a primate, a rodent, a livestock animal, or a game animal. Preferably the primate is a human.

In some specific embodiments, the effective amount is an amount of Akkermansia muciniphila MG784 in the range of about 1×10² cfu/day to about 1×10¹⁵ cfu/day.

The present invention also provides a method for improving constipation, enhancing immune function, protecting the intestine, regulating the intestinal microbiota, discharging oil, reducing fat, providing antioxidant effects, providing anti-inflammatory effects, or inhibiting acetylcholinesterase activity, characterized in that the method comprises administering to a subject an effective amount of the aforementioned Akkermansia muciniphila MG784 or the aforementioned composition.

In some specific embodiments, the subject can be a primate, a rodent, a livestock animal, or a game animal. Primates include chimpanzees, cynomolgus monkeys, spider monkeys, macaques, or humans. Rodents include mice, rats, marmots, ferrets, rabbits, and hamsters. Domestic and game animals include cattle, horses, pigs, deer, bison, buffalo, felines such as domestic cats, canines such as dogs, foxes, wolves, and avian species. Preferably, the subject is a human.

In some specific embodiments, the effective amount can be in the range of 1×10² cfu/day to about 1×10¹⁵ cfu/day, preferably about 1×10⁴ cfu/day to about 1×10¹² cfu/day, more preferably about 1×10⁵ cfu/day to about 1×10¹⁰ cfu/day, and even more preferably about 1×10⁶ cfu/day to about 1×10⁹ cfu/day.

In another embodiment of the present invention, the daily amount of Akkermansia muciniphila administered daily is in the range of 1×10⁶ cfu/day to about 1×10¹⁰ cfu/day, preferably about 1×10⁸ cfu/day to about 1×10¹⁰ cfu/day, more preferably about 1×10⁹ cfu/day to about 1×10¹⁰ cfu/day.

In another embodiment of the present invention, the daily amount of Akkermansia muciniphila administered daily is in the range of 1×10⁶ cfu/day to about 1×10¹⁰ cfu/day, preferably about 1×10⁶ cfu/day to about 1×10⁹ cfu/day, more preferably about 1×10⁸ cfu/day to about 1×10⁹ cfu/day.

In the present invention, the term “Akkermansia muciniphila” refers to the strictly anaerobic mucin-degrading bacterium identified by Derrien (Derrien et al., International Journal of Systematic and Evolutionary Microbiology, 2004, 54:1469-1476). The cells are oval, non-motile, and Gram-stain-negative. Akkermansia muciniphila may also be referred to as a species of the genus Akkermansia or an Akkermansia-like bacterium. It belongs to the Chlamydiae/Verrucomicrobia group; phylum Verrucomicrobia. If the taxonomy changes, those skilled in the art will understand how to adapt to the changes in taxonomy to infer strains useful in the present invention. Strains having a genomic similarity of about 70% are generally considered to be the same species.

In the present invention, the term “fermentation product” refers to cellular components, metabolites, secreted molecules and compounds, and the like, metabolized by Akkermansia muciniphila. The fermentation product can be obtained, for example, by recovering the supernatant of an Akkermansia muciniphila culture or by extracting cellular components or cell fractions, metabolites, or secreted compounds from an Akkermansia muciniphila culture. Fermentation product can also refer to degradation products. Fermentation product may correspond to components in isolated form from Akkermansia muciniphila, or any mixture of one or more components from Akkermansia muciniphila.

In the present invention, the term “about” preceding a number means the value of the number plus or minus 20%, preferably 10%.

In the present invention, the term “similarity” is defined as the percentage of nucleotide residues in a candidate nucleotide sequence that are identical to the nucleotide residues in a reference nucleotide sequence, after aligning the nucleotide sequences (and introducing gaps if necessary) to achieve the maximum percentage sequence identity, and not considering any conservative substitutions as part of sequence identity. Sequence alignment can be performed using various methods known in the art to determine percentage nucleotide sequence similarity, for example, using publicly available computer software such as BLAST, BLAST-2, ALIGN, or MEGALIGN (DNASTAR) software. Those skilled in the art can determine the appropriate parameters for measuring alignment, including any algorithms necessary to obtain the maximum alignment over the full length of the sequences being compared.

In the present invention, the term “vitamin” refers to a class of trace organic substances that humans and animals must obtain from food to maintain normal physiological functions. Common examples can be selected from one or more of vitamin A, vitamin B complex, vitamin C, vitamin D, vitamin E, or vitamin K.

In the present invention, the term “mineral” refers to inorganic elements essential for the human body, often existing in the form of metal salts, such as calcium salts, phosphates, potassium salts, sodium salts, magnesium salts, iron salts, zinc salts, copper salts, iodates, or selenates.

In the present invention, the term “probiotic” refers to live microorganisms which, when administered in adequate amounts, confer a health benefit on the host, for example, restoring or improving the composition of the intestinal microbial flora. They are often provided as dietary supplements containing potentially beneficial bacteria or yeasts and are widely used in foods, including dairy products and probiotic-fortified foods.

In the present invention, the term “oil-discharging or fat-reducing” may be understood as an auxiliary physiological process that reduces fat absorption by blocking partial dietary fat from being absorbed by the human body in the intestine, thereby allowing it to be directly excreted. This process operates through the following two physiological pathways: 1) by inhibiting the activity of fat-digesting enzymes (such as pancreatic lipase) in the intestine, thereby preventing dietary fat from being broken down into absorbable small molecules. As a result, undigested macromolecular fat cannot penetrate the intestinal wall and is excreted directly via feces, reducing fat absorption; 2) by physically adsorbing or encapsulating fat particles in the gut to form large molecular complexes that cannot be absorbed, likewise leading to fecal excretion of fat and reduced lipid absorption.

In the present invention, the term “effective amount” is defined as an amount effective to achieve the desired biological result (e.g., reducing, preventing, or treating a disease or condition and/or inducing a specific beneficial effect) at the necessary dosage and for the necessary period of time. The effective amount of the composition of the present disclosure can vary depending on factors such as the age, sex, and weight of the individual. The dosing regimen can be adjusted to provide the optimal response. Several divided doses may be administered daily, or the dose may be proportionally reduced as indicated by the exigencies of the individual situation. As will be readily understood, the composition according to the present disclosure can be administered in a single portion or in multiple portions at intervals throughout the day. As those skilled in the art will appreciate, the portions need not be limited to daily administration and can be on a convenient and effective basis such as every two days or every three days. Depending on the exigencies of the situation, administration on a given day may be in a single portion or multiple portions at intervals throughout the day.

The following specific examples illustrate the embodiments of the present invention. Those skilled in the art can easily understand other advantages and effects of the present invention from the content disclosed in this specification. The present invention can also be implemented or applied through other different specific embodiments, and various details in this specification can be modified or changed based on different viewpoints and applications without departing from the spirit of the present invention.

Before further describing the specific embodiments of the present invention, it should be understood that the scope of protection of the present invention is not limited to the specific embodiments described below; it should also be understood that the terms used in the examples of the present invention are for describing specific embodiments and are not intended to limit the scope of protection of the present invention; in the specification and claims of the present invention, unless otherwise expressly stated, the singular forms “a,” “an,” and “the” include plural referents.

When a numerical range is given in an example, it should be understood that, unless otherwise stated in the present invention, any numerical value between the two endpoints of the range and the two endpoints themselves can be selected. Unless otherwise defined, all technical and scientific terms used herein have the same meaning as commonly understood by one of ordinary skill in the art to which this invention belongs. In addition to the specific methods, equipment, and materials used in the examples, any methods, equipment, and materials of the prior art similar or equivalent to those described in the examples of the present invention can be used to implement the present invention based on the prior art knowledge possessed by those skilled in the art and the records of the present invention.

The nucleotide sequence information used in this application is as follows:

16S rDNA sequence of Akkermansia muciniphila MG784

SEQ ID No. 1
CTCGATAGAGAGCTTATAGACTGCAGTCGACGAGAGATTGCTTAGCTTGC
TAATAATTCTCTAGTGGCGCACGGGTGAGTAACACGTGAGTAACCTGCCC
CCGAGAGCGGGATAGCCCTGGGAAACTGGGATTAATACCGCATAGTATCG
AAAGATTAAAGCAGCAATGCGCTTGGGGATGGGCTCGCGGCCTATTAGTT
AGTTGGTGAGGTAACGGCTCACCAAGGCGATGACGGGTAGCCGGTCTGAG
AGGATGTCCGGCCACACTGGAACTGAGACACGGTCCAGACACCTACGGGT
GGCAGCAGTCGAGAATCATTCACAATGGGGGAAACCCTGATGGTGCGACG
CCGCGTGGGGGAATGAAGGTCTTCGGATTGTAAACCCCTGTCATGTGGGA
GCAAATTAAAAAGATAGTACCACAAGAGGAAGAGACGGCTAACTCTGTGC
CAGCAGCCGCGGTAATACAGAGGTCTCAAGCGTTGTTCGGAATCACTGGG
CGTAAAGCGTGCGTAGGCTGTTTCGTAAGTCGTGTGTGAAAGGCGCGGGC
TCAACCCGCGGACGGCACATGATACTGCGAGACTAGAGTAATGGAGGGGG
AACCGGAATTCTCGGTGTAGCAGTGAAATGCGTAGATATCGAGAGGAACA
CTCGTGGCGAAGGCGGGTTCCTGGACATTAACTGACGCTGAGGCACGAAG
GCCAGGGGAGCGAAAGGGATTAGATACCCCTGTAGTCCTGGCAGTAAACG
GTGCACGCTTGGTGTGCGGGGAATCGACCCCCTGCGTGCCGGAGCTAACG
CGTTAAGCGTGCCGCCTGGGGAGTACGGTCGCAAGATTAAAACTCAAAGA
AATTGACGGGGACCCGCACAAGCGGTGGAGTATGTGGCTTAATTCGATGC
AACGCGAAGAACCTTACCTGGGCTTGACATGTAATGAACAACATGTGAAA
GCATGCGACTCTTCGGAGGCGTTACACAGGTGCTGCATGGCCGTCGTCAG
CTCGTGTCGTGAGATGTTTGGTTAAGTCCAGCAACGAGCGCAACCCCTGT
TGCCAGTTACCAGCACGTGAAGGTGGGGACTCTGGCGAGACTGCCCAGAT
CAACTGGGAGGAAGGTGGGGACGACGTCAGGTCAGTATGGCCCTTATGCC
CAGGGCTGCACACGTACTACAATGCCCAGTACAGAGGGGGCCGAAGCCGC
GAGGCGGAGGAAATCCTGAAAACTGGGCCCAGTTCGGACTGTAGGCTGCA
ACCCGCCTACACGAAGCCGGAATCGCTAGTAATGGCGCATCAGCTACGGC
GCCGTGAATACGTTCCCGGGTCTTGTACACACCGCCCGTCACATCATGGA
AGCCGGTCGCACCCGAAGTATCTGAAGCCAACCGCAAGGAGGCAGTCATA
AGGAGACATTG

### Example 1 Isolation and Culture of Akkermansia muciniphila MG784

Strain MG784 was isolated from the intestine of a healthy Chinese infant. Its 16S rDNA sequence information is shown as SEQ ID NO. 1, and its identification result is Akkermansia muciniphila. Strain MG784 was cryopreserved at −80° C. in glycerol stock vials. Generally, it can be inoculated on the surface of BHI+mucin solid medium plates and incubated inverted at 37° C. under anaerobic conditions for 2-8 days to obtain colonies. A single colony was picked and inoculated into BHI+mucin liquid medium and incubated at 37° C. under anaerobic conditions for 12-96 hours to obtain an activated strain. The BHI+mucin liquid medium can be prepared by adding an appropriate amount of mucin to BHI (e.g., catalog number HB8297-1), such as mixing 30 g BHI medium with 1 g mucin dissolved in 1 L water; BHI+mucin solid medium is the BHI+mucin liquid medium with an appropriate amount of agar to solidify the liquid medium.

### Example 2 Inhibitory Effect of Akkermansia muciniphila MG784 on Acetylcholinesterase Activity

Akkermansia muciniphila MG84 cultured in BHI liquid medium as described in Example 1 to the stationary phase was harvested to obtain a bacterial suspension. After pasteurization treatment, inactivated bacterial liquid was obtained. The inhibitory ability of the inactivated bacterial liquid on acetylcholinesterase (AchE) activity was then measured using an Acetylcholinesterase Inhibitor Screening Kit (manufacturer: Shanghai Haling Biotechnology Co., Ltd., catalog number: HL70017.6). The control strain Akkermansia muciniphila BNCC341917 was purchased from BNCC.

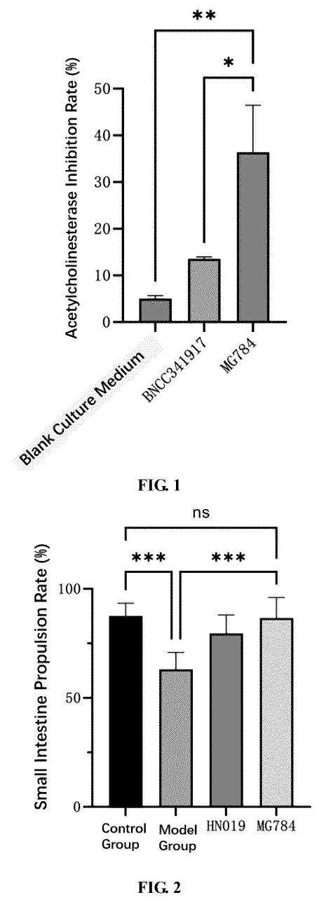

The above experimental results are shown in FIG. 1. The inactivated Akkermansia muciniphila MG784 significantly inhibited acetylcholinesterase activity compared to the commercially available Akk strain (Akkermansia muciniphila BNCC341917), with an inhibition rate of about 36.42%, indicating that the inactivated bacteria of Akkermansia muciniphila MG784 can effectively inhibit acetylcholinesterase activity.

### Example 3 Use of Akkermansia muciniphila MG784 for Improving Constipation

Preparation of bacterial powder: A purified MG784 single colony was picked and inoculated into 10 mL of BHI medium, cultured anaerobically at 37° C. for 12-96 hours; Activated culture was transferred to fresh BHI medium, expanded under the same conditions, then transferred to BHI medium for further anaerobic culture for 48 hours; The bacterial culture was collected, pasteurized, centrifuged to collect the bacterial pellet, followed by freeze-drying, grinding, and sieving to prepare bacterial powder.

After one week of adaptive feeding, 100 Balb/c mice were randomly divided into 4 groups. The administration groups were gavaged with the corresponding doses. The blank group and the model group were gavaged with distilled water. After 7 days of gavage, except for the normal control group, the other groups were subjected to modeling. Each group of mice was divided into two subgroups, subgroup A (10 mice) and subgroup B (15 mice). In the morning, loperamide hydrochloride (10 mg/kg) was administered by gavage for 7 days. In the afternoon, gavage administration was performed according to the corresponding doses. The control group and the normal group were gavaged with pure water. The AKK784 group was administered 2.0×10⁹ cfu/mouse/day.

### 3.1 Small Intestine Propulsion Rate

Subgroup A mice were fasted for 12-16 hours. The model group and each dose group were then gavaged with loperamide hydrochloride, while the control group was gavaged with an equal amount of physiological saline. After 30 minutes, each group was gavaged with ink. After completing the gavage, 20 minutes later, the mice were sacrificed by cervical dislocation. The small intestine was taken and laid flat on a scale ruler. The distance from the pylorus to the ink front (L1) and the distance from the pylorus to the cecum (L2) were measured to calculate the intestinal propulsion rate. Small intestine propulsion rate=L1/L2×100%.

The above results are shown in FIG. 2. Compared with the control group, the small intestine propulsion rate of the model group mice was significantly reduced, indicating successful construction of the constipation-induced intestinal propulsion model. Compared with the model group, the small intestine propulsion rate of the AKK784 group was significantly increased.

### 3.2 Improvement in Fecal Parameters (Pellets Count, Weight, Etc.)

Subgroup B mice were treated similarly to subgroup A. After fasting for 12-16 hours, the model group and each dose group were gavaged with loperamide hydrochloride, while the control group was gavaged with an equal amount of physiological saline. After 30 minutes, each group was gavaged with ink. Fresh feces were collected for 6 hours, every 2 hours. The time to the first black stool, the total number of fecal pellets, and the fecal weight over 6 hours were recorded.

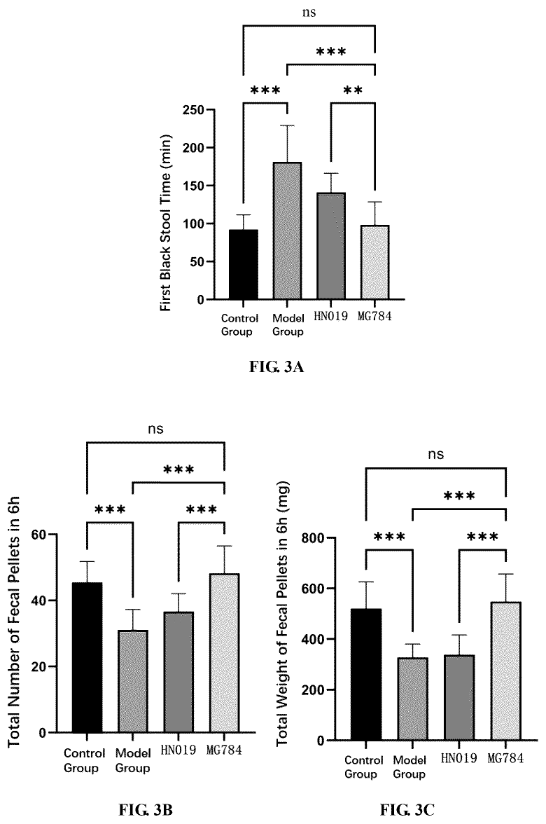

The above results are shown in FIGS. 3A-3C. Compared with the control group, the model group showed a significantly increased time to the first defecation, a significantly reduced total number of fecal pellets, and a significantly reduced 6-hour fecal weight. Compared with the model group and the positive control group, the AKK784 group showed a significantly reduced time to the first defecation, a significantly increased total number of fecal pellets, and a significantly increased 6-hour fecal weight.

### 3.3 Detection of Biochemical Indicators (Motilin (MTL), Gastrin (GAS), Acetylcholine, Etc.)

The day after fecal observation for subgroup B mice, the mice were subjected to orbital blood sampling. Serum was separated, aliquoted, and stored at −20° C. for measuring serum levels of motilin (MTL) and gastrin (GAS). A 2 cm section of mouse colon tissue was flash-frozen in liquid nitrogen and stored at −80° C. for measuring acetylcholine content in the colon.

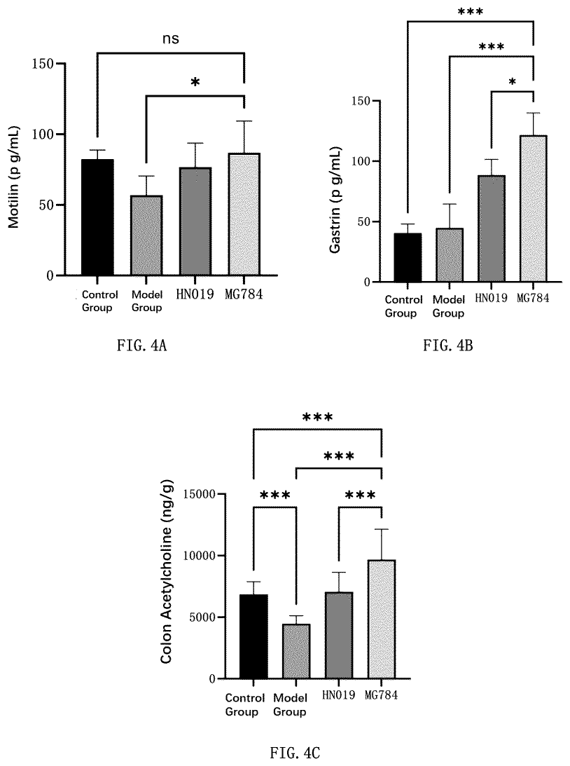

The above results are shown in FIGS. 4A-4C. Compared with the control group, the excitatory neurotransmitter motilin (MTL) was decreased in the model group, while gastrin (GAS) levels showed no significant change. Compared with the model group and the positive control group, MG AKK784 showed a trend of increased motilin (MTL) and significantly increased gastrin (GAS) levels.

### Example 4 Use of Akkermansia muciniphila MG784 for Protecting the Intestine

A 2 cm section of colon tissue was taken from subgroup A mice, preserved in fixative solution, embedded in paraffin overnight, and paraffin sections of colon tissue were prepared. Subsequently, HE staining was performed. Histopathological changes in the colon tissue were observed under an optical microscope and photographed for preservation.

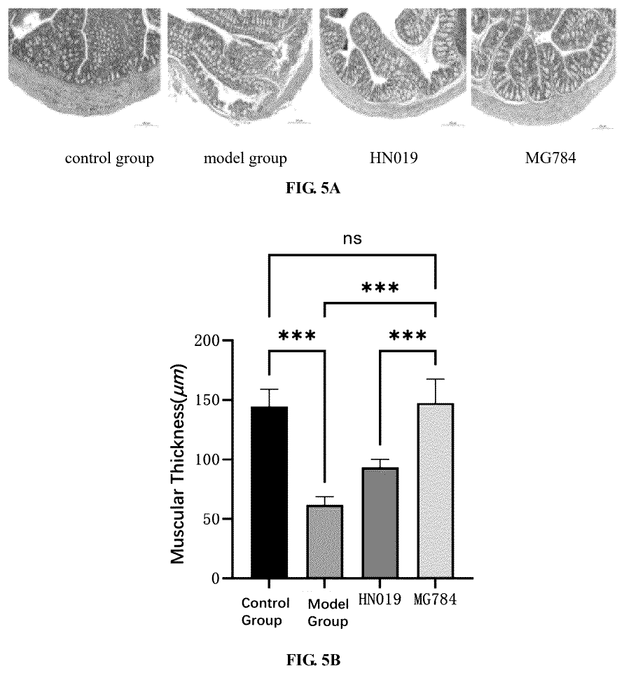

The above results are shown in FIGS. 5A-5B. The colon mucosa structure of the control group mice was intact, with glands arranged neatly and epithelial cells exhibiting normal morphology; the smooth muscle fibers of the muscular layer were arranged orderly with uniform thickness. The colon tissue of the model group mice showed significant inflammatory reactions in the mucosa, disordered gland arrangement, and irregular epithelial cell morphology, which could affect intestinal secretion and absorption functions; this was accompanied by thinning of the intestinal muscular layer. Thinning of the muscular layer may interfere with normal intestinal peristalsis, slowing the transit of intestinal contents and thereby affecting digestion and absorption functions. Compared with the model group, the positive control group and the AKK MG784 group showed varying degrees of reduction in colonic mucosal inflammation and relatively neat gland arrangement. Among them, AKK MG784 significantly improved inflammatory cell infiltration and significantly increased the thickness of the muscular layer.

### Example 5 Use of Akkermansia muciniphila MG784 for Regulating the Intestinal Microbiota

Fecal samples from the ileocecal region of subgroup A mice were collected into sterile cryotubes. Operations were performed rapidly, minimizing contamination during the process. All samples were immediately placed in liquid nitrogen after collection and stored at −80° C. after sampling for subsequent 16S sequencing. The abundance of beneficial bacteria (Akkermansia, Bifidobacterium, Lacticaseibacillus (formerly Lactobacillus cheese group), Bacteroides, Lactobacillus) and harmful bacteria (Streptococcus) in the intestinal microbiota of mice from each group was detected.

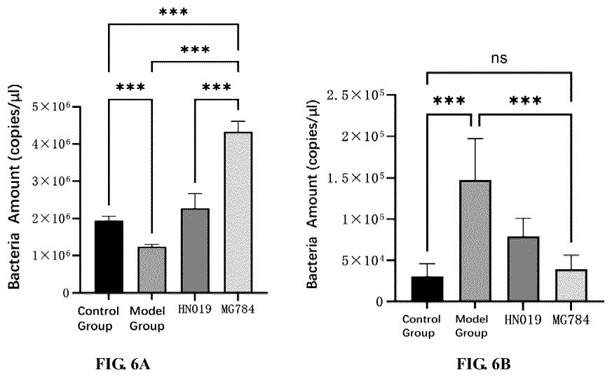

The above experimental results are shown in FIGS. 6A-6B. Compared with the model group, the positive control group and the AKK MG784 group significantly increased the amount of beneficial bacteria; Streptococcus is a harmful intestinal bacterial genus. Compared with the model group, the positive control group and the AKK MG784 group reduced the abundance of the Streptococcus genus to varying degrees.

In summary, the Akkermansia muciniphila strain MG784 provided by the present invention, as an intestinal probiotic, can increase the levels of motilin, gastrin, and acetylcholine in the gastrointestinal tract, promote intestinal peristalsis, improve the small intestine propulsion rate, and ameliorate defecation time and fecal output. Furthermore, Akkermansia muciniphila MG784 can alleviate colonic mucosal inflammation, improve the thickness of the digestive tract muscular layer, modulate the structure of the intestinal microbiota, significantly increase the abundance of beneficial bacteria, and decrease the abundance of harmful bacteria.

The above examples are intended to illustrate the embodiments disclosed in the present invention and should not be construed as limiting the invention. In addition, the various modifications and variations in the methods of the invention mentioned herein will be obvious to those skilled in the art without departing from the scope and spirit of the invention. Although the present invention has been described in detail with reference to various specific preferred embodiments, it should be understood that the invention should not be limited to these specific embodiments. In fact, various modifications obvious to those skilled in the art, as described above, for obtaining the invention should be included within the scope of the present invention.

What is claimed is:

1. An Akkermansia muciniphila MG784, wherein the Akkermansia muciniphila MG784 has the deposition number DSM 35549.

2. The Akkermansia muciniphila MG784 according to claim 1, wherein the Akkermansia muciniphila MG784 comprises a DNA fragment having a similarity of 50% or more to the nucleotide sequence shown as SEQ ID No. 1.

3. The Akkermansia muciniphila MG784 according to claim 2, wherein the Akkermansia muciniphila MG784 comprises a DNA fragment having a nucleotide sequence similarity of 50%, 55%, 60%, 65%, 70%, 75%, 80%, 82%, 84%, 86%, 88%, 90%, 91%, 92%, 93%, 94%, 95%, 96%, 97%, 98%, or 99% or more to SEQ ID NO. 1.

4. A composition comprising the Akkermansia muciniphila MG784 according to claim 1.

5. The composition according to claim 4, wherein the composition further comprises one or more selected from the group consisting of probiotic, prebiotic, vitamin, mineral, amino acid, polypeptide, emulsifier, thickener, sweetener, flavoring agent, stabilizer, and preservative.

6. The composition according to claim 5, wherein the probiotic is selected from one or more of Bifidobacterium, Lactobacillus, Enterococcus, Bacillus, Clostridium, Saccharomyces, Weizmannia (formerly Bacillus), or Lactococcus.

7. The composition according to claim 6, wherein the probiotic is one or more selected from the group consisting of Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium longum, Bifidobacterium infantis, Bifidobacterium animalis, Bifidobacterium bifidum, Bifidobacterium adolescentis, Bifidobacterium lactis, Enterococcus faecalis, Enterococcus faecium, Lactococcus lactis, Streptococcus salivarius, Saccharomyces cerevisiae and Saccharomyces boulardii, Clostridium butyricum, Bacillus subtilis, Bacillus indicus, Bacillus licheniformis, Bacillus clausii, or Bacillus coagulans.

8. The composition according to claim 4, wherein the prebiotic is one or more selected from the group consisting of inulin, inulin-type fructan, fructo-oligosaccharides, xylose, arabinose, arabinoxylans, ribose, galactose, rhamnose, cellobiose, fructose, lactose, salicin, sucrose, glucose, esculin, Tween 80, trehalose, maltose, mannose, melibiose, mucus or mucin, raffinose, fructo-oligosaccharides, galacto-oligosaccharides, or amino acid.

9. The composition according to claim 4, wherein the Akkermansia muciniphila strain MG784 has a concentration in the composition of at least 1×10⁹ CFU/g, at least 2×10⁹ CFU/g, at least 3×10⁹ CFU/g, at least 4×10⁹ CFU/g, at least 5×10⁹ CFU/g, at least 6×10⁹ CFU/g, at least 7×10⁹ CFU/g, at least 8×10⁹ CFU/g, at least 9×10⁹ CFU/g, at least 1×10¹⁰ CFU/g, at least 2×10¹⁰ CFU/g, at least 3×10¹⁰ CFU/g, at least 4×10¹⁰ CFU/g, at least 5×10¹⁰ CFU/g, at least 6×10¹⁰ CFU/g, at least 7×10¹⁰ CFU/g, at least 8×10¹⁰ CFU/g, at least 9×10¹⁰ CFU/g, or at least 1×10¹¹ CFU/g or more.

10. The composition according to claim 4, wherein the composition is selected from one or more of capsule, tablet, powder, granule, powder, oral liquid, drop, gummy, pressed candy, jelly, solid beverage, or fermented dairy product.

11. Use of the Akkermansia muciniphila MG784 according to claim 1, or the fermentation product thereof, for improving constipation, a product for enhancing immune function, a product for protecting the intestine, a product for regulating the intestinal microbiota, a product for oil-discharging or fat-reducing, an antioxidant product, an anti-inflammatory product, or an acetylcholinesterase inhibitor.

12. The use according to claim 11, wherein the product for improving constipation, the product for enhancing immune function, the product for regulating the intestinal microbiota, the product for oil-discharging or fat-reducing, the antioxidant product, or the anti-inflammatory product is a food, health food, or pharmaceutical.

13. The use according to claim 11, wherein the product or inhibitor further comprises an excipient acceptable for food, health food, or pharmaceutical.

14. The use according to claim 11, wherein the product for improving constipation has one or more of the following functions:
1) enhancing intestinal peristalsis;
2) reducing defecation time;
3) increasing fecal weight;
4) increasing intestinal acetylcholine content.

15. The use according to claim 11, wherein the product for enhancing immune function is a product for improving intestinal immunity.

16. The use according to claim 11, wherein the product for protecting the intestine is a product for improving intestinal barrier function.

17. The use according to claim 11, wherein the use comprises administering to a subject an effective amount of the Akkermansia muciniphila MG784 or its fermentation product.

18. The use according to claim 17, wherein the subject is a primate, a rodent, a livestock animal, or a game animal.

19. The use according to claim 18, wherein the primate is a human.

20. The use according to claim 17, wherein the effective amount is an amount of Akkermansia muciniphila MG784 in the range of about 1×10² cfu/day to about 1×10¹⁵ cfu/day.
